(19) United States
(12) Patent Application Publication
(10) Pub. No.: US 2005/0203340 A1
(43) Pub. Date: Sep. 15, 2005

(54) Steerable colonoscope probe with variable stiffness

(76) Inventors: John Butler, Deansgrange (IE); Frank Bonadio, Bray (IE); Shane Joseph MacNally, Bray (IE)

Correspondence Address: KUDIRKA & JOBSE, LLP, ONE STATE STREET, SUITE 800, BOSTON, MA, 02109, US

(21) Appl. No.: 10/935,477

(22) Filed: Sep. 7, 2004

Related U.S. Application Data
(63) Continuation of application No. PCT/IE03/00036, filed on Mar. 6, 2003.

(30) Foreign Application Priority Data
Mar. 6, 2002 (IE) 020170

(51) Int. Cl. A61B 1/01

(52) U.S. Cl. 600/114; 600/144; 600/146

(57) ABSTRACT

An assembly for advancing a colonoscope through the colon comprises a colonoscope 2, a tube 4 and means for steering the tube 4. The steering means comprises a series of wires 20 extending along the tube 4 to the tube distal end 5 to which the wires 20 are attached. By pulling on one or more wires 20 from a proximal end of the tube 4, the tube distal end 4 may be steered by an operator as desired. The stiffness of the colonoscope 2 may be increased or decreased in use.

This invention relates to an assembly and a method for advancing a probe through a lumen. In particular it relates to an assembly and a method for advancing a colonoscope through a colon.

During conventional colonoscopy procedures a colonoscope is advanced through the tortuous sigmoid colon until the colonoscope reaches the descending colon. The colonoscope is then manipulated to reduce the redundancy in the sigmoid colon. When the sigmoid colon has been straightened, the colonoscope is typically further advanced through the colon.

However, this type of procedure is generally difficult to perform, and/or painful for the patient due to stretching of the colon which occurs upon impact between the colonoscope and the wall of the colon as the colonoscope is advanced, especially during advancement of the colonoscope around the bends of the tortuous sigmoid colon.

This invention is aimed at overcoming this problem.

### STATEMENTS OF INVENTION

According to the invention, there is provided a method of advancing a colonoscope through a colon, the method comprising the steps of:—
a) providing a colonoscope;
b) providing a tube;
c) inserting the colonoscope into a colon;
d) advancing the colonoscope until the colonoscope encounters a bend in the colon;
e) advancing the tube over the colonoscope;
f) steering the tube around the bend in the colon; and
g) advancing the colonoscope out of the tube beyond the bend.

Because the tube can be steered around a bend in a colon, the method and assembly of the invention enables a colonoscope to be advanced through a colon while minimising or avoiding contact between the colonoscope and the wall of the colon, even in the case of a tortuous colon.

In one embodiment of the invention the steps d) to g) are repeated at least once to advance the colonoscope through the colon in an incremental manner.

In another case the tube is advanced over the colonoscope until the tube distal end is substantially aligned with the colonoscope distal end.

The tube may be steered around the bend by manipulating the tube. Alternately the tube is steered around the bend by manipulating the colonoscope.

In a preferred embodiment of the invention the method comprises the step of altering in situ the stiffness of the colonoscope and/or the tube so that the colonoscope is stiff relative to the tube before advancing the tube over the colonoscope. By altering the stiffness in situ so that the colonoscope is stiff relative to the tube during advancement of the tube over the colonoscope, the colonoscope acts as a stiff guide to guide advancement of the tube and minimise contact between the tube and the colon wall.

The method may comprise the step of altering in situ the stiffness of the colonoscope and/or the tube so that the colonoscope is flexible relative to the tube before advancing the colonoscope out of the tube. By altering the stiffness in situ so that the colonoscope is flexible relative to the tube during advancement of the colonoscope out of the tube, the tube acts as a stiff guide to guide advancement of the colonoscope and minimise contact between the colonoscope and the colon wall.

The tube may be steered around the bend after altering the stiffness so that the colonoscope is flexible relative to the tube.

The stiffness of the colonoscope may be altered. In another embodiment the stiffness of the tube is altered.

The invention minimises impact between the colonoscope and the colon wall, thus minimising stretching of the colon and the resultant discomfort or pain experienced by the patient during such a colonoscopy procedure, and also making the colonoscopy procedure easier to perform.

In one application, the invention enables a colonoscopist to advance a colonoscope through a sigmoid colon without stretching of the sigmoid colon being caused.

It will be appreciated that it is not essential that the steps of the method be performed in a certain order. For example, the tube may be steered around a bend in a colon before or after altering the stiffness so that the colonoscope is flexible relative to the tube.

In another aspect of the invention, there is provided an assembly for advancing a colonoscope through a lumen, the assembly comprising:—
a colonoscope;
a tube having a colonoscope lumen extending therethrough for passing the tube over the colonoscope; and
means for steering the tube.

In one embodiment the tube comprises the means for steering.

In another embodiment the colonoscope comprises the means for steering, the colonoscope being engagable with the tube to steer the tube.

The assembly may comprise means for altering the stiffness of the colonoscope and/or the tube between a first mode in which the colonoscope is stiff relative to the tube, and a second mode in which the colonoscope is flexible relative to the tube.

The colonoscope may comprise the means for altering the stiffness of the colonoscope. Preferably the tube is of constant stiffness. The tube may be pliable.

In another case the tube may comprise the means for altering the stiffness of the tube.

In one embodiment the colonoscope comprises means to view a lumen distally of the colonoscope.

The viewing means further assist in avoiding contact between the assembly and a colon wall.

The invention also provides in another aspect a probe for advancing through a lumen, the probe comprising:—
means for steering the probe; and
means for altering the stiffness of the probe between a first mode in which the probe is relatively stiff, and a second mode in which the probe is relatively flexible.

According to a further aspect of the invention, there is provided a tube having a probe lumen extending therethrough for passing the tube over a probe, the tube comprising:—
means for steering the tube; and
means for altering the stiffness of the tube between a first mode in which the tube is relatively flexible, and a second mode in which the tube is relatively stiff.

In this specification the term bend will be understood to mean any geometrical configuration of the lumen, and/or any matter present in the lumen whether attached to the lumen wall or otherwise, which acts to hinder passage of the probe and/or the tube through the lumen.

### BRIEF DESCRIPTION OF THE DRAWINGS

The invention will be more clearly understood from the following description of an embodiment thereof, given by way of example only, with reference to the accompanying drawings in which:—

FIG. 1 is a schematic view illustrating advancing a colonoscope through a colon;

FIG. 2 is a schematic view illustrating advancing a tube over the colonoscope;

FIG. 3 is a schematic view illustrating steering the tube around a bend in the lumen;

FIG. 4 is a schematic view illustrating advancing the colonoscope out of the tube;

FIG. 5 is a schematic view illustrating advancing the tube over the colonoscope;

FIG. 6 is a schematic view illustrating steering the tube around a bend in the colon;

FIG. 7 is a schematic view illustrating advancing the colonoscope out of the tube;

FIGS. 8(a) to 8(e) are schematic views illustrating advancing a colonoscope through a colon;

FIG. 9 is perspective view of a steerable tube used in the invention, in one configuration of use; and

FIG. 10 is a perspective view of the tube of FIG. 9 in another configuration of use.

### DETAILED DESCRIPTION

Referring to the drawings, there is illustrated an assembly according to the invention for advancing a colonoscope probe through a colon.

The assembly comprises a colonoscope probe 2, having a distal end 3, a tube 4 having a distal end 5 and a proximal end 19, means for steering the tube 4, and means for altering the stiffness of the colonoscope 2 and/or the tube 4.

The tube 4 has a probe lumen 6 extending through the tube 4 to facilitate passing the tube 4 over the colonoscope 2.

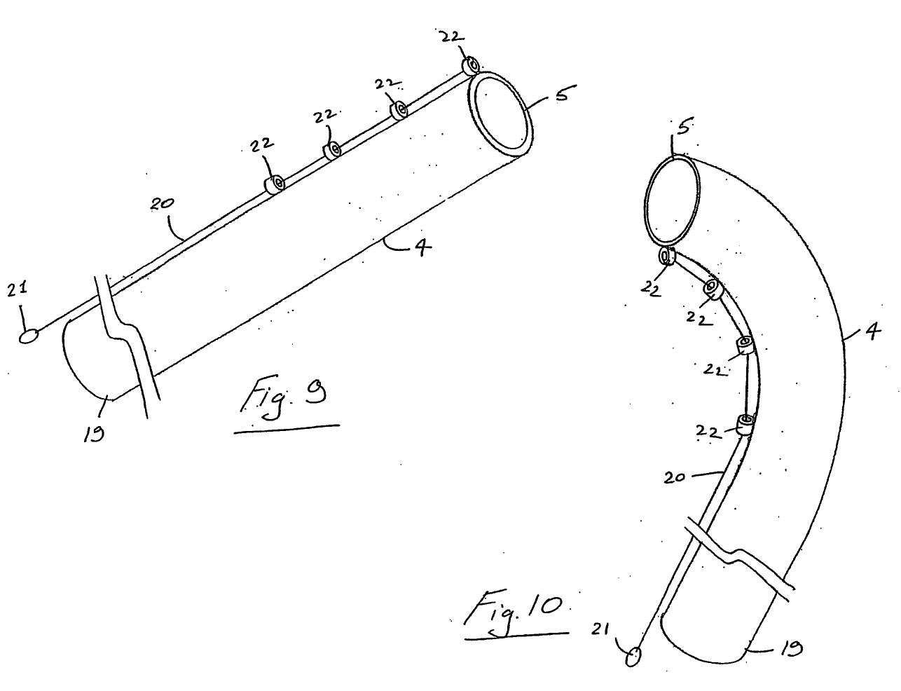

In this case, the tube 4 comprises the means for steering the tube 4. As illustrated in FIGS. 9 and 10 the steering means may be provided by one or more wires 20 extending along the tube 4 to the tube distal end 5 to which the wires 20 are attached. By pulling on a handle 21 on one or more wires 20 from a proximal end of the tube 4, the tube distal end 5 may be steered by an operator as desired. The wires 20 are shown exposed on the outside of the tube for illustrative purposes. They may be in a sheath and/or may be located in the wall of or on the inside or outside of the tube 4.

In the tube 4 of FIGS. 9 and 10 a single wire 20 is provided extending through eyelets 22. This simple version facilitates steering in one direction which is the plane of the eyelets. Adding another linear series of eyelets and associated steering wire will allow further steering flexibility in another plane.

The colonoscope probe 2 may be of an energy actuated polymer material, such that upon application of energy, in this case a particular voltage difference across the colonoscope 2, the stiffness of at least portion of the colonoscope 2 may be increased or decreased. The stiffness of the tube 4, in this case, remains constant, the tube 4 being pliable.

In a first mode the colonoscope 2 is stiff relative to the tube 4 to facilitate advancement of the tube 4 over the colonoscope 2 (FIG. 2), and in a second mode the colonoscope 2 is flexible relative to the tube 4 to facilitate advancement of the colonoscope 2 out of the probe lumen 6 (FIG. 4).

The colonoscope 2 has means to view a lumen, such as a colon 7, distally of the colonoscope distal end 3. The viewing means is provided by a fibre optic cable extending through the colonoscope 2.

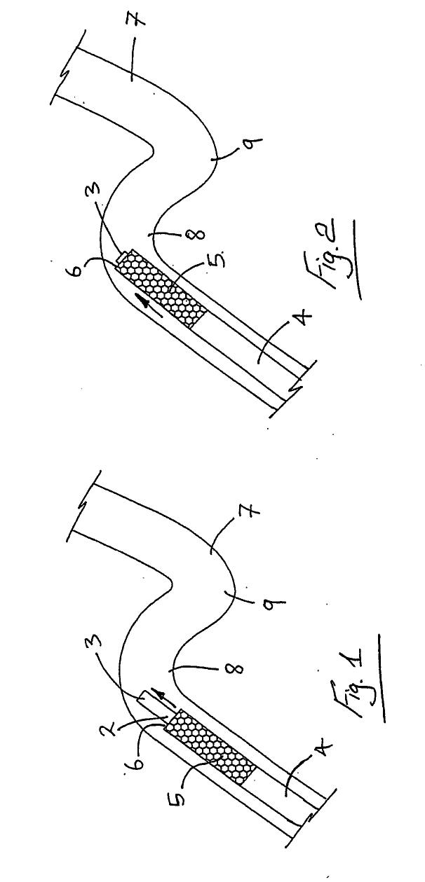

In use, the colonoscope 2 is inserted into the colon 7, and advanced through the colon 7 until the colonoscope distal end 3 encounters a bend 8 in the colon 7, (FIG. 1) or a large polyp on the colon wall, or some other obstruction to further advancement of the colonoscope 2.

The stiffness of the colonoscope 2 is then altered in situ by applying a voltage difference across the colonoscope 2 so that the assembly is in the first mode and the colonoscope 2 is stiff relative to the tube 4. The tube 4 is advanced over the stiff colonoscope 2 until the tube distal end 5 is substantially aligned with the colonoscope distal end 3 (FIG. 2). Because the colonoscope 2 is stiff relative to the tube 4 during advancement of the tube 4 over the colonoscope 2, the colonoscope 2 acts as a stiff guide to guide advancement of the tube 4. In this way impact between the tube 4 and the colon 7 is minimised, and stretching of the bend 8 of the colon 7 into a loop is avoided.

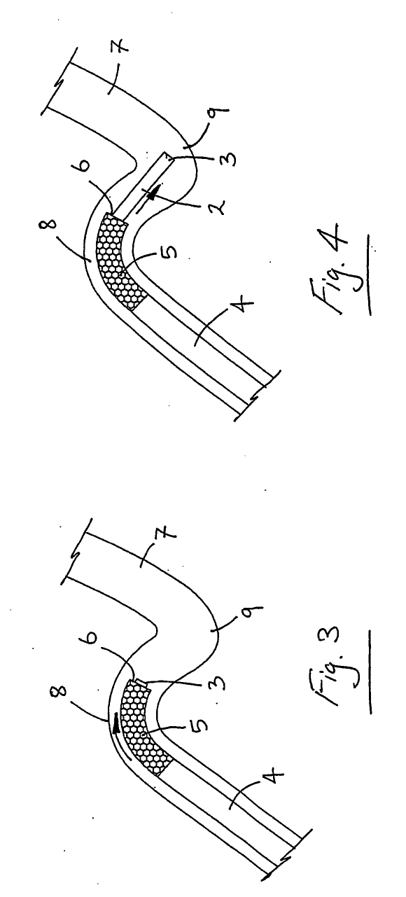

By applying a voltage difference across the colonoscope 2, the stiffness of the colonoscope 2 is altered again in situ this time to the second mode so that the colonoscope 2 is flexible relative to the tube 4, and the tube 4 is manipulated to steer the tube distal end 5 around the bend 8 in the colon 7 (FIG. 3). By advancing the flexible colonoscope 2 out of the probe lumen 6, the colonoscope 2 passes around the bend 8 and further through the colon 7 (FIG. 4). Because the colonoscope 2 is flexible relative to the tube 4 during advancement of the colonoscope 2 out of the tube 4, the tube 4 acts as a stiff guide to guide advancement of the colonoscope 2. In this way impact between the colonoscope 2 and the colon 7 is minimised, and stretching of the bend 8 of the colon 7 into a loop is avoided.

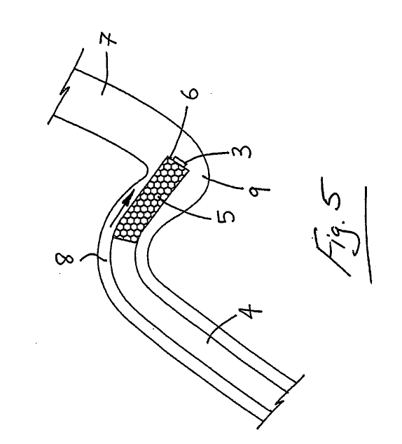

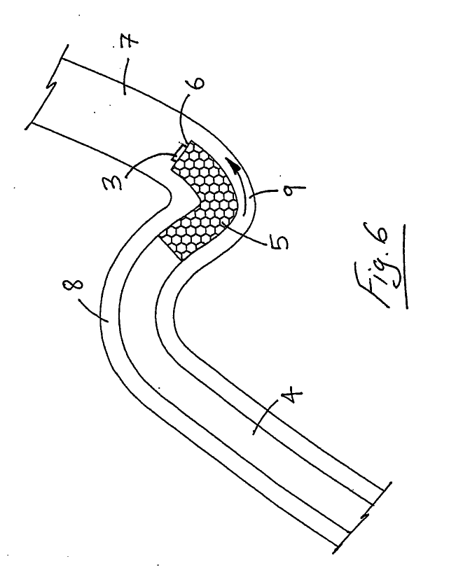

The colonoscope 2 is advanced until the colonoscope distal end 3 encounters a second bend 9 in the colon 7. To pass the colonoscope 2 around the second bend 9, the procedure described previously with reference to FIGS. 1 to 4 is repeated. In particular, the colonoscope 2 is stiffened, and the tube 4 is advanced over the stiff colonoscope 2 (FIG. 5). The colonoscope 2 is then made flexible, the tube distal end 5 is steered around the second bend 9 (FIG. 6), and the flexible colonoscope 2 is advanced out of the probe lumen 6 (FIG. 7).

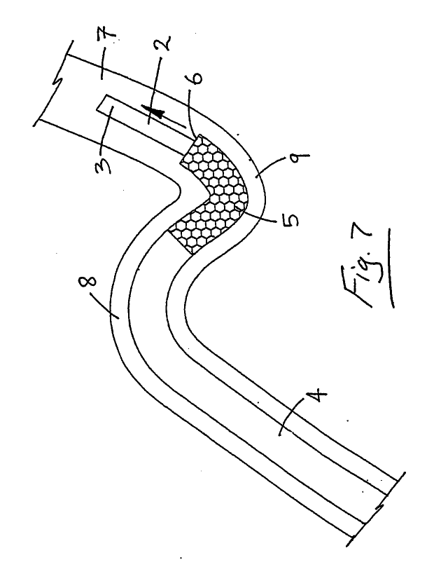

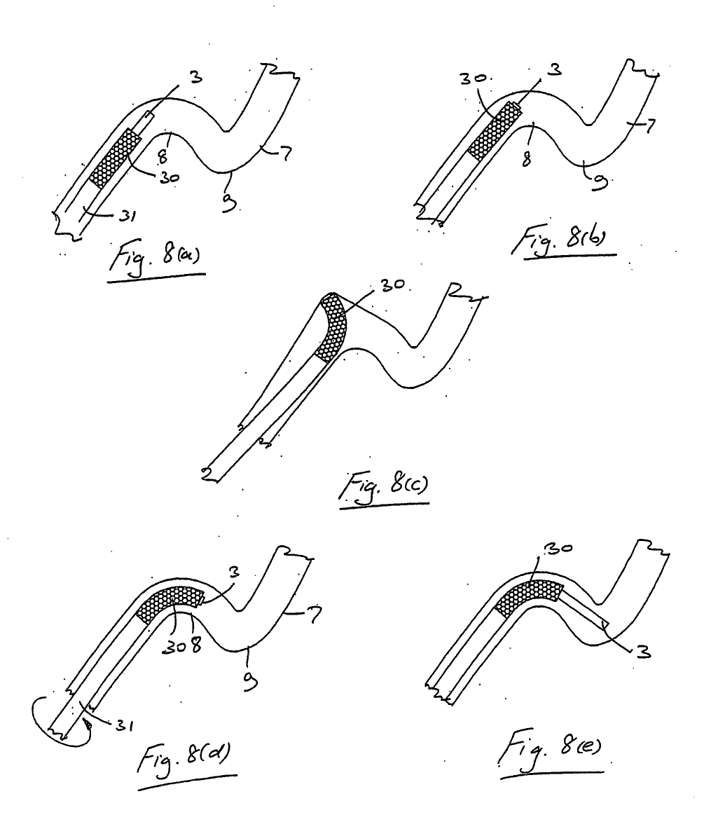

Referring to FIGS. 8(a) to 8(e) there is illustrated another assembly for advancing a colonoscope through a colon in which parts similar to those of FIGS. 1 to 7 are assigned the same reference numerals. In this case a bending section 30 is configured to bend in one direction. Activating a steering mechanism will generally cause the overtube tip 30 to embed in the colon wall. The body of the overtube 31 is then rotated until the bend is correctly aligned in the lumen of the colon. The probe may then be advanced and the method repeated at the next bend encountered.

It will be appreciated that this procedure for advancing a colonoscope beyond bends and/or other obstructions in a colon may be repeated as many times as necessary to advance the colonoscope through the colon in an incremental manner.

It will further be appreciated that the colonoscope probe may comprise means for steering in addition to or as an alternative to the steering means on the tube. In such a case, the probe may be manipulated to engage the probe distal end with the tube, and thereby steer the tube around a bend in a lumen, before altering the stiffness so that the probe is flexible relative to the tube.

The steering means may be provided in any suitable form. Energy actuated polymers could be used to steer the colonoscope and/or the tube.

The stiffness of the tube may be altered in situ in addition to or as an alternative to altering the stiffness of the colonoscope in situ, provided that in the first mode the colonoscope is stiff relative to the tube and in the second mode the colonoscope is flexible relative to the tube.

Any suitable means for altering the stiffness of the colonoscope probe and/or the tube may be employed. For example, a series of wires may be provided extending along the colonoscope to the distal end to which the wires are attached. By manipulation of one or more of the wires, the colonoscope probe stiffness may be altered. A similar arrangement could be used for altering the stiffness of the tube.

Although the assembly and method of use thereof have been described in detail in relation to advancement of a colonoscope through a colon, it will be appreciated that the invention may be used for advancement of a probe through any lumen, such as a vasculature, or a tortuous pipe network, or a small-scale cable duct.

The invention is not limited to the embodiments hereinbefore described, with reference to the accompanying drawings, which may be varied in construction and detail.

1. An assembly for advancing a colonoscope probe through a lumen, the assembly comprising:
a colonoscope probe;
a tube having a colonoscope lumen extending therethrough for passing the tube over the colonoscope; and
means for steering the tube.

2. An assembly as claimed in claim 1 wherein the tube comprises the means for steering.

3. An assembly as claimed in claim 1 wherein the probe comprises the means for steering, the probe being engagable with the tube to steer the tube.

4. An assembly as claimed in claim 1 wherein the assembly comprises means for altering the stiffness of at least one of: the colonoscope and the tube between a first mode in which the colonoscope is stiff relative to the tube, and a second mode in which the colonoscope is flexible relative to the tube.

5. An assembly as claimed in claim 4 wherein the colonoscope comprises the means for altering the stiffness of the colonoscope.

6. An assembly as claimed in claim 5 wherein the tube is of constant stiffness.

7. An assembly as claimed in claim 6 wherein the tube is pliable.

8. An assembly as claimed in claim 4 wherein the tube comprises the means for altering the stiffness of the tube.

9. An assembly as claimed in claim 1 wherein the colonoscope comprises means to view a lumen distally of the colonoscope.

10. (canceled)

11. A probe for advancing through a lumen, the probe comprising:
means for steering the probe; and
means for altering stiffness of the probe between a first mode in which the probe is relatively stiff, and a second mode in which the probe is relatively flexible.

12. A tube having a probe lumen extending therethrough for passing the tube over a probe, the tube comprising:
means for steering the tube; and
means for altering stiffness of the tube between a first mode in which the tube is relatively flexible, and a second mode in which the tube is relatively stiff.

13. A method of advancing a colonoscope through a colon, the method comprising:
a) providing a colonoscope;
b) providing a tube;
c) inserting the colonoscope into a colon;
d) advancing the colonoscope until the colonoscope encounters a bend in the colon;
e) advancing the tube over the colonoscope;
f) steering the tube around the bend in the colon; and
g) advancing the colonoscope out of the tube beyond the bend.

14. A method as claimed in claim 13 wherein the steps d) to g) are repeated at least once to advance the colonoscope through the colon in an incremental manner.

15. A method as claimed in claim 13 wherein the tube is advanced over the colonoscope until a tube distal end is substantially aligned with a colonoscope distal end.

16. A method as claimed in claim 13 wherein the tube is steered around the bend by manipulating at least one of: the tube and the colonoscope.

17. (canceled)

18. A method as claimed in claim 13 further comprising altering, in situ, stiffness of at least one of the colonoscope and the tube so that the colonoscope is stiff relative to the tube before advancing the tube over the colonoscope.

19. A method as claimed in claim 13 further comprising altering, in situ, stiffness of at least one of the colonoscope and the tube so that the colonoscope is flexible relative to the tube before advancing the colonoscope out of the tube.

20. A method as claimed in claim 19 wherein the tube is steered around the bend after altering the stiffness so that the colonoscope is flexible relative to the tube.

21. A method as claimed in claim 18 wherein the stiffness of at least one of the colonoscope and the tube is altered.

22. (canceled)

23. (canceled)

24. An assembly, comprising:
a colonoscope probe;
a tube having a colonoscope lumen for slidably receiving the colonoscope probe therein; and
a steering mechanism to position the assembly within a body lumen.

25. The assembly as recited in claim 24, wherein:
the steering mechanism is coupled to at least one of the tube and the probe.

26. The assembly as recited in claim 24, wherein the steering mechanism is coupled to the tube and the steering mechanism comprises:
at least one wire extending along the tube to a tube distal end.

27. The assembly as recited in claim 24, wherein the probe comprises:
energy actuated polymer material.

28. The assembly as recited in claim 24, further comprising at least one of:
a first mechanism to alter a stiffness characteristic of the probe; and
a second mechanism to alter a stiffness characteristic of the tube.
